(19) United States
(12) Patent Application Publication
(10) Pub. No.: US 2015/0141768 A1
(43) Pub. Date: May 21, 2015

(54) Smart Fiber-Optic Sensor System and Method for Optical Spectroscopy in Robotic Surgical Systems

(71) Applicants: Bing Yu, Hudson, OH (US); Erik D. Engeberg, Cuyahoga Falls, OH (US)

(72) Inventors: Bing Yu, Hudson, OH (US); Erik D. Engeberg, Cuyahoga Falls, OH (US)

(73) Assignee: THE UNIVERSITY OF AKRON, Akron, OH (US)

(21) Appl. No.: 14/523,520

(22) Filed: Oct. 24, 2014

Related U.S. Application Data
(60) Provisional application No. 61/895,148, filed on Oct. 24, 2013.

(51) Int. Cl. A61B 1/07; A61B 5/107; A61B 19/00; A61B 1/313; A61B 5/00; A61B 5/01

(52) U.S. Cl. CPC A61B 1/07; A61B 5/0084; A61B 5/6843; A61B 5/015; A61B 19/2203; A61B 1/3132; A61B 5/4836; A61B 5/7203; A61B 5/0075; A61B 5/1076; A61B 2019/2215
USPC 600/301; 606/130

(57) ABSTRACT

A smart fiber-optic sensor system for use with robotic surgical systems performs optical spectroscopy using a diffuse reflectance spectroscopy (DRS) sensing channel, a self-calibration (SC) channel, a pressure-sensing channel, and a temperature sensing channel. During use of the fiber-optic sensor during a laparoscopic procedure, the pressure-sensing channel ensures that the fiber-optic sensor is maintained in suitable contact with the target tissue being treated. In addition, the temperature sensor is used to ensure that the target tissue does not exceed a desired temperature from the use of an electrosurgical cutting device during the laparoscopic procedure, so as to prevent burning or charring damage at the target tissue.

### CROSS-REFERENCE TO RELATED APPLICATION

This application claims the benefit of U.S. Provisional Application No. 61/895,148 filed on Oct. 24, 2013, the content of which is incorporated herein by reference.

### TECHNICAL FIELD

Generally, the present invention relates to fiber-optic sensors for quantitative optical spectroscopy. In particular, the present invention is directed to a smart fiber-optic sensor system for optical spectroscopy of biological tissue, for use in robot-assisted laparoscopic (RAL) procedures. Particularly, the present invention is directed to a smart fiber-optic sensor system for RAL procedures, which includes a temperature sensor to prevent charring and collateral damage to the biological tissue being treated by laparoscopic electrosurgical devices.

### BACKGROUND ART

Robot-assisted laparoscopy (RAL) is an emerging, minimally-invasive surgical (MIS) technique that combines the advantages of both laparoscopy and robotic surgery. In addition, RAL systems, such as the da Vinci Surgical System®, represent the latest innovations in the field of MIS technology. Thus, because of the desirable benefits of MIS to the patient, such as reduced trauma to the surgical site and reduced post-operative recovery time, the number of laparoscopic and robot-assisted surgeries performed annually continues to increase, as highly invasive or “open” surgical procedures are converted to MIS procedures.

Robot-assisted laparoscopy (RAL) combines the benefits of minimal invasiveness provided by laparoscopic techniques with the features of robotic surgery, which includes providing multiple degrees of motion at the robotic end-effector that carries the surgical cutting instrument; providing 3-dimensional views, decreasing the fatigue and tension tremor of the surgeon when controlling the laparoscopic instrument; and providing improved dexterity and precision when executing the surgical procedure. Although RAL has only recently been adopted in the field of surgery, it has been increasingly adopted for use in the fields of gynecologic oncology, urology, cardiac surgery, liver resection, pancreatic surgery, prostatectomy, and general surgery.

Despite improvements over the years, clinically relevant problems still exist with laparoscopic electrosurgical tissue dissection or cutting devices, such as those used by RAL systems. For example, surgeons are often unable to prevent collateral damage to tissue at or near the surgical site due to heat that is generated by the uncontrolled spread of energy from the flow of electrical current that is delivered to the surgical site by the electrosurgical device used to perform robot-assisted laparoscopic (RAL) procedures. In particular, the heat energy that is generated from the electrosurgical cutting instrument generally results in the overheating, charring, and tearing of the tissue near or proximate to the surgical site, which can complicate the surgical procedure and lengthen patient recovery time. Furthermore, due to the nature of laparoscopic procedures, such as robot-assisted laparoscopic procedures, the field of view provided to a surgeon when completing the procedure is small, and as a result, tissue burns typically go unnoticed when they occur. The effects of these unnoticed burns may remain latent, such that the impact on the health of the patient is not realized until after the operation is complete, at which time diagnosis becomes more difficult. Moreover, it is estimated that patients have been burned over 230,000 times every year due to the use of electrosurgical equipment during laparoscopic procedures, while complications during electrosurgery on the small and large intestine, bile duct and during the performance of hysterectomies have been reported at clinically relevant rates. Injuries to ovaries have also been documented during electrosurgical treatment of polycystic ovary syndrome.

Furthermore, surgeons have requested that robot-assisted laparoscopic (RAL) systems provide additional information regarding real-time tissue diagnosis, which can assist the surgeon in deciding what and where to cut during surgery. In response, numerous imaging techniques, such as MRI, fluorescence, confocal microscopy and optical coherence tomography, have been applied to assist surgeons during a robot-assisted surgical procedure to visualize blood vessels, tumors and nerves. Among these imaging modalities, fluorescence imaging has been pursued for incorporation into various commercial systems, such as the da Vinci® surgical system. However, such imaging methods are generally limited to providing high-resolution structural information, but little diagnostic information regarding the physiology of the tissue being investigated or treated. Furthermore, few attempts have been made to obtain quantitative information about the characteristics and physiology of biological tissue, as well as biological tumor tissue that is manipulated or treated by a robot-assisted laparoscopy (RAL) surgical system.

Optical spectroscopy, such as fiber-optic-based diffuse reflectance spectroscopy (DRS), is a nondestructive and noninvasive technique that provides quantitative information of biological tissue in-vivo, using the optical properties of the tissue, such as wavelength-dependent light absorption and light-scattering properties of the tissue. That is, DRS provides various information regarding the biological tissue based on the light absorption and light scattering properties imparted by the tissue. For example, light scattering in biological tissue is affected by the local inhomogeneities in the refractive index of the tissue, whereby such inhomogeneities include cellular organelles of the tissue and the extracellular matrix of the tissue. Such inhomogeneities, which cause light scattering in biological tissue, have been found to change with carcinogenesis and tumor necrosis. The amount of light absorption of biological tissue is primarily the result of hemoglobin concentrations, which allows for quantitative characterization of tissue perfusion and oxygenation (SO₂). As such, quantitative DRS has recently been used for pre-cancer detection and cancer diagnostics, intra-operative tumor margin assessment, monitoring of tumor response to therapy, and tissue oximetry, as well as surgical guidance. Thus, it would be desirable to have a robotic fiber-optic instrument (RFOI) device that utilizes DRS to provide real-time tissue diagnosis to facilitate surgeons in deciding what to cut (e.g. malignant vs. normal/benign tissue) and where to cut, so as to reduce undesired tissue damages during a robot-assisted laparoscopic (RAL) procedure.

Therefore, there is a need for a smart fiber-optic sensor system that provides quantitative optical spectroscopy for robot-assisted laparoscopic (RAL) surgical systems in accordance with the concepts of the present invention. In addition, there is a need for a smart fiber-optic sensor system for robot-assisted laparoscopic (RAL) surgical systems that includes an interferometric temperature carried by a robotic end-effector to identify the temperature of the tissue being treated during laparoscopic procedures to prevent tissue damage. There is also a need for a smart fiber-optic sensor system that integrates a diffuse reflectance spectroscopy (DRS) channel, a self-calibration channel, a fiber-optic pressure, and a temperature sensor into a single probe or instrument for use in a robot-assisted laparoscopic system to provide quantitative tissue characteristics, diagnosis and temperature of the tissue in real time during the laparoscopic procedure. In addition, there is a need for a smart fiber-optic sensor system that carries out robot-assisted laparoscopy (RAL) procedures with a reduced amount of tissue damage in the form of burning and charring, so as to reduce the overall cost of the surgical procedure and patient recovery time, while lessening the risk the surgeon assumes in performing such procedures.

### SUMMARY OF THE INVENTION

In light of the foregoing, it is a first aspect of the present invention to provide a smart fiber optic sensor comprising a sensing channel for illuminating a specimen and for collecting spectral reflections from the specimen from which specimen spectral data can be determined; a pressure sensing channel for collecting pressure sensor spectral reflections from which a contact pressure can be determined; a temperature sensing channel for collecting temperature sensor spectral reflections from which a temperature of the specimen can be determined; and a calibration channel for obtaining calibration spectral reflections usable for correcting the specimen spectral data.

A further aspect of the present invention is to provide a method for utilizing a smart fiber optic sensor for measuring a specimen, the method comprising contacting the specimen with the smart fiber optic sensor; generating, using the smart fiber optic sensor, specimen spectral data, pressure sensor spectral data, temperature sensor spectral data and calibration spectral data; calculating a contact pressure at an interface of the smart fiber optic sensor and the specimen using the pressure sensor spectral data; calculating a temperature at the interface using the temperature sensor spectral data; and correcting specimen spectral data using the calibration spectral data.

The subject matter described herein comprises smart fiber-optic sensors, systems, and methods for quantitative tissue optical spectroscopy for robot-assisted surgical systems. In one embodiment, the system can include a smart fiber-optic sensor comprising a specimen sensing channel, a self-calibrating channel, a pressure-sensing channel, and a temperature-sensing channel, which operate concurrently and in real time to prevent heat-related damage to the target tissue during a robot-assisted laparoscopic (RAL) procedure.

### BRIEF DESCRIPTION OF THE DRAWINGS

These and other features and advantages of the present invention will become better understood with regard to the following description, appended claims, and accompanying drawings wherein:

FIG. 1 is a block diagram of a smart fiber-optic sensor system for optical spectroscopy in accordance with the concepts of the present invention;

FIG. 2 shows the source and detection fiber optics for use with the smart fiber-optic sensor system in accordance with the concepts of the present invention;

FIGS. 3A and 3B are cross-sectional views of an embodiment of the smart fiber-optic sensor system in accordance with the concepts of the present invention;

FIGS. 4A and 4B are cross-sectional views of an alternative embodiment of the smart fiber-optic sensor system in accordance with the concepts of the present invention;

FIG. 5 is a cross-sectional view of a pressure and temperature sensor provided by the smart fiber-optic sensor system in accordance with the concepts of the present invention;

FIG. 6 is a block diagram of the pressure sensor in accordance with the concepts of the present invention;

FIG. 7 is a cross-sectional view of a temperature sensor provided by the smart fiber-optic sensor system in accordance with the concepts of the present invention;

FIG. 8 is a graph showing test results of the temperature sensor provided by the smart fiber-optic sensor system, whereby the sensor achieves an accuracy of about +/−0.1° C. within a temperature range of about 10-100° C.;

FIG. 9 is a perspective view of a robotic end-effector of a robotic surgical system, such as a robot-assisted laparoscopy (RAL) system for carrying a probe tip provided by the smart fiber-optic sensor system in accordance with the concepts of the present invention; and

FIG. 10 is a flow diagram showing the operational steps taken by the smart fiber-optic sensor system using the temperature sensor during a robot-assisted laparoscopic procedure in accordance with the concepts of the present invention.

### DETAILED DESCRIPTION OF THE INVENTION

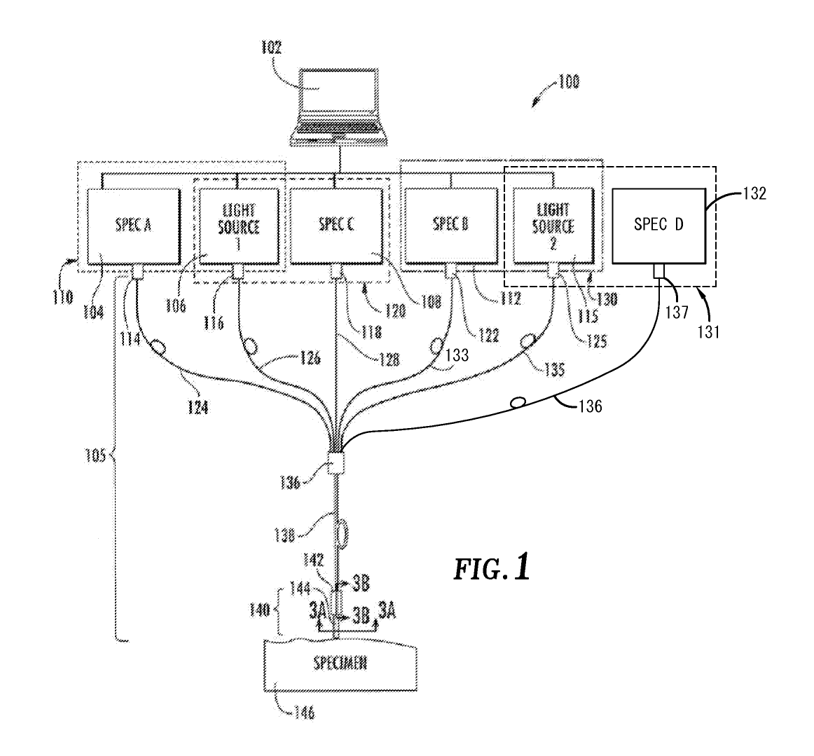

A smart fiber-optic sensor system for use with robotic surgical systems, such as robot-assisted laparoscopy (RAL) systems is generally referred to by numeral 100, as shown in FIG. 1 of the drawings. The smart fiber-optic sensor system 100 comprises a diffuse reflectance spectroscopy (DRS) system, to be discussed, which allows the system 100 to perform noninvasive UV-Vis-NIR (ultraviolet-visible-near infrared) diffuse reflectance spectroscopy, as well as fluorescence spectroscopy of any desired biological tissue. The system 100 may comprise one or more light emitting diodes (LEDs) as illumination sources, a four-channel spectrometer for light detection, a fiber-optic probe, and a processing unit or module for processing custom computer readable media. The smart fiber-optic sensor system 100 may also integrate a diaphragm-based Fabry-Perot interferometric (DFPI) pressure sensor and/or a Fabry-Perot interferometric temperature sensor with a tissue-sensing channel. Thus, the smart fiber-optic system 100 may be configured for use with robotic surgical systems, such as robot-assisted laparoscopy systems, which will be discussed in detail below.

As shown in FIG. 1, the smart fiber-optic sensor system 100 comprises a processor or processing unit 102, which may include any suitable portable or standalone computing device. The processing unit 102 is connected via a wired or wireless interface to each of the four fiber-optic groupings or “channels” 110, 120, 130, 131, and a smart fiber-optic probe or sensor, generally designated 105. It should also be appreciated that each of the channels may comprise a fiber-optic grouping or “legs” that are distributed between various illumination sources, imaging spectrometers and/or spectrometer channels A, B, C, and D, and the probe or sensor 105. Additionally, one or more of the fiber-optic channels can share an illumination source and/or an imaging spectrometer or spectrometer channel A, B, C, D.

A first fiber-optic channel 110 comprises a diffuse reflectance spectroscopy (DRS) sensing channel 110 that includes a first light source 106 and a first spectrometer channel A, designated 104. A second fiber-optic channel 120 comprises a self-calibration channel 120, wherein the illumination source comprises the first light source 106 and a second spectrometer channel C, designated 108. Thus, both the DRS-sensing channel 110 and the self-calibration channel 120 are configured to share the first light source 106. Optionally, the self-calibration channel 120 may utilize the same spectrometer and/or spectrometer channel as either of the sensing channel 110 or a fiber-optic pressure sensor channel to be discussed.

A third fiber-optic channel 130 comprises a fiber-optic pressure sensor channel 130, which includes a second light source 115 and a third spectrometer channel B, designated 112.

A fourth fiber-optic channel 131 comprises a fiber-optic temperature sensor channel 131, which includes the second light source 115 and a fourth spectrometer channel D, designated 132. In one aspect, the temperature-sensing channel 131 may comprise an interferometric temperature sensor, which is able to provide real-time temperature data at the probe-specimen interface, in a manner to be discussed.

As such, the reflectance and/or fluorescence spectrum from the specimen of target biological tissue, designated as 146, can be detected by the first spectrometer channel A 104; the calibration spectrum can be detected by the second spectrometer channel C 108; the spectrum from a pressure sensor can be detected by the third spectrometer channel B 112; and the spectrum from the temperature sensor can be detected by the fourth spectrometer channel D 132.

In an alternative embodiment, three separate spectrometers may be used and shared among the fiber-optic channels rather than using a four-channel spectrometer. In another alternative embodiment, a dual-channel spectrometer may be used instead of a three-channel spectrometer, whereby each spectrometer comprises a dual-channel fiber-optic spectrometer, such as those manufactured by Avantes BV, wherein one channel could detect DRS from the sensing channel, and the second spectrometer channel is shared by each of the calibration channel 120, the pressure-sensing channel 130, and the temperature-sensing channel 131. That is, the second channel of a dual-channel spectrometer could detect the signals from the calibration channel, the pressure sensor, and the temperature sensor. In one embodiment the spectrometers may comprise a white light emitting diode (LED) based miniature spectrometer comprising a high-power, white LED and a USB 4000 spectrometer, such as those manufactured by Ocean Optics of Orlando, Fla., in one aspect, the spectrometers may comprise a 1-mm optical fiber for illumination and another 1-mm optical fiber for collection with a source-to-detector separation (SDS) of 2.3 mm.

The fiber-optic pressure sensor channel 130 may comprise an interferometric pressure sensor, which may provide real-time pressure data at the interface of the probe tip 144 and the specimen 148, such that an operator or technician can manually control the pressure applied by the tip 144 of the robotic surgical system at the interface within an optimal minimal range. It should be appreciated that the robotic surgical system may be configured to take corrective action automatically based on the pressure detected by the pressure sensor channel 130. This ensures optimal specimen coupling between the probe tip 144 and the specimen 146, so that the surgical robot does not apply unnecessary levels of pressure on the sensor tip 144 that could affect the physiology of the biological tissue specimen 146.

In another embodiment, the fiber-optic temperature sensor channel 131 may comprise an interferometric temperature sensor, which provides real-time temperature data at the interface of the probe tip 144 and the tissue specimen 146. As a result, the operator of the robot-assisted surgical system can identify the temperature of the tissue specimen 146, so that an operator of the robotic surgical system can control the electrosurgical device (or through automated corrective action at the robotic surgical system), such as an ultrasound device, used during the laparoscopic surgical procedure in a manner to prevent burning and charring of the tissue.

Thus, the smart fiber-optic sensor or probe 105 may be configured to integrate or combine the DRS-sensing channel 110, the calibration channel 120, the interferometric pressure-sensing channel 130, and/or the interferometric temperature-sensing channel 132 into a single optical probe. Furthermore, the smart fiber-optic sensor 105 may be configured for use in connection with any probe instrument, including but not limited to, side-firing and forward-firing probes.

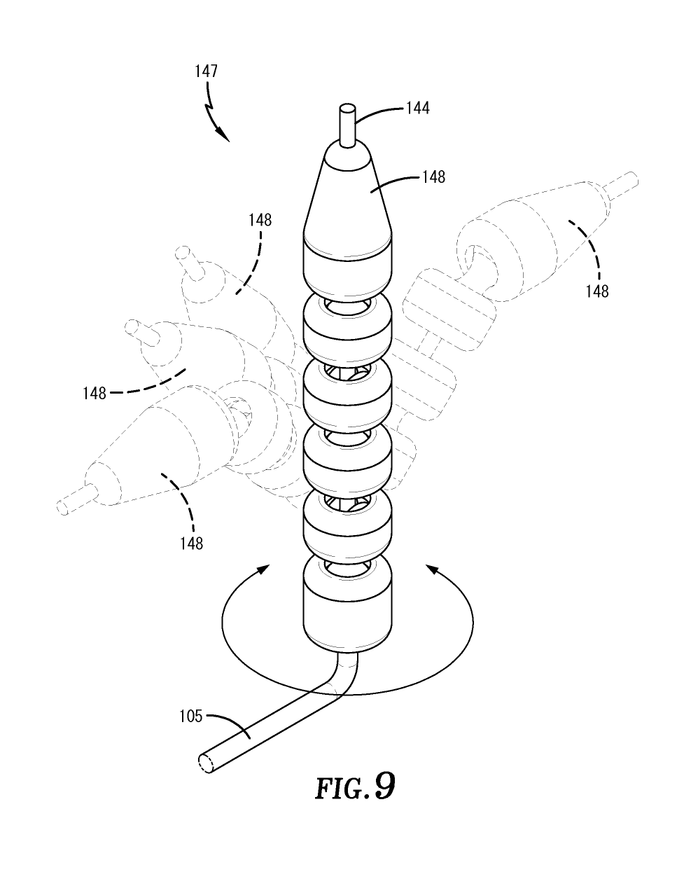

It should also be appreciated that the smart fiber-optic sensor 105 is configured to be utilized with a robotic surgical system 147, shown in FIG. 9, which may comprise any desired robotic surgical system, such as a robot-assisted laparoscopic (RAL) surgical system. That is, the sensor or probe 105 may be attached to or made integral with an articulating end-effector 147 of a robot-assisted laparoscopy (RAL) surgical system. In one aspect, the end-effector 148 may be configured to move the probe tip 144 of the sensor 105 in any desired direction through any degree of motion and rotation, so as to move the probe tip 144 in any desired position in three dimensions.

With regard to the DRS-sensing channel 110, it may comprise a detection fiber portion, or collection leg 124, which is coupled to the first spectrometer 104 at coupler 114. The DRS-sensing channel 110 may also comprise an illumination fiber leg 126 that is coupled to the first light source 108 at coupler 116 for collecting DRS data from the biological tissue specimen 146, such as an in-vivo tissue specimen, for example.

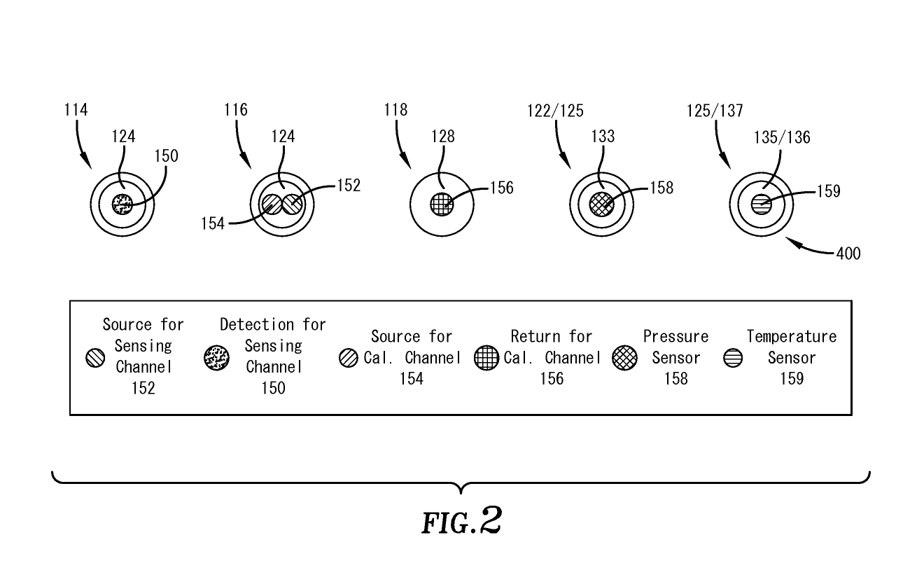

In one embodiment, the first light source 106 may comprise a white light source, which uses white light emitting diodes (LEDs), such as white LEDs, LE-1x-c manufactured by WT&T Inc., for example. The light source 106 may also generate light having a wavelength, which is in the range of about 400 to 700 nm, which serves as the light source for the DRS-sensing channel 110. In one embodiment, the DRS-sensing channel 110 may comprise a high-power white LED as the light source for DRS and/or one or multiple UV (ultraviolet)/visible LEDs (with or without a bandpass filter) as the excitation source for fluorescence spectroscopy. The white LED and color LED(s) may share the same light source fibers (152, FIG. 2) or have independent source fibers. The fluorescence channel may be used to quantify both intrinsic fluorophores (such as FAD, NADH, collagen, and porphyrin), and endogenous fluorophores (such as the nanoparticle contrast agents, chemotherapy drug, and Doxirubicin). The different LEDs may be switched on/off sequentially, in other embodiments, the first light source 106 may comprise any suitable broadband light source. In one embodiment, a broadband light source with a monochromator (e.g., a scanning double-excitation monochromator) or a plurality of laser diodes may also be used along with a plurality of photo-detectors in lieu of spectrometer 104.

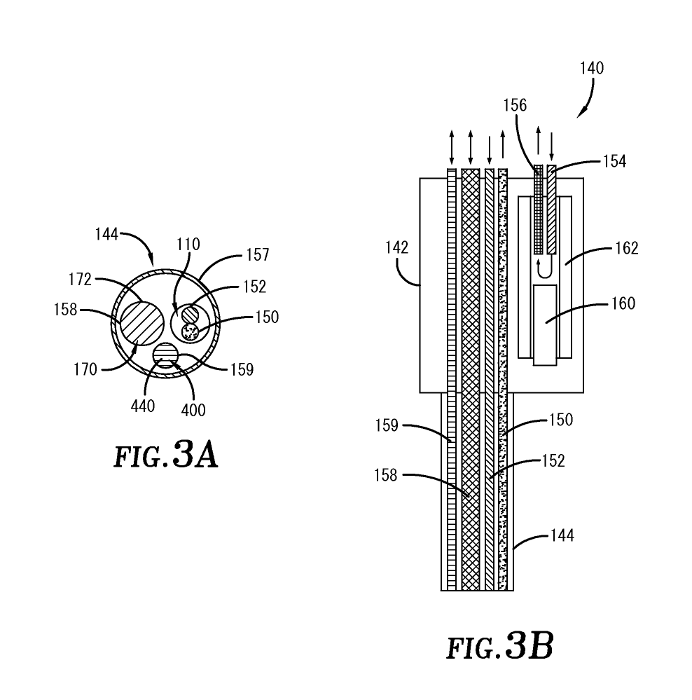

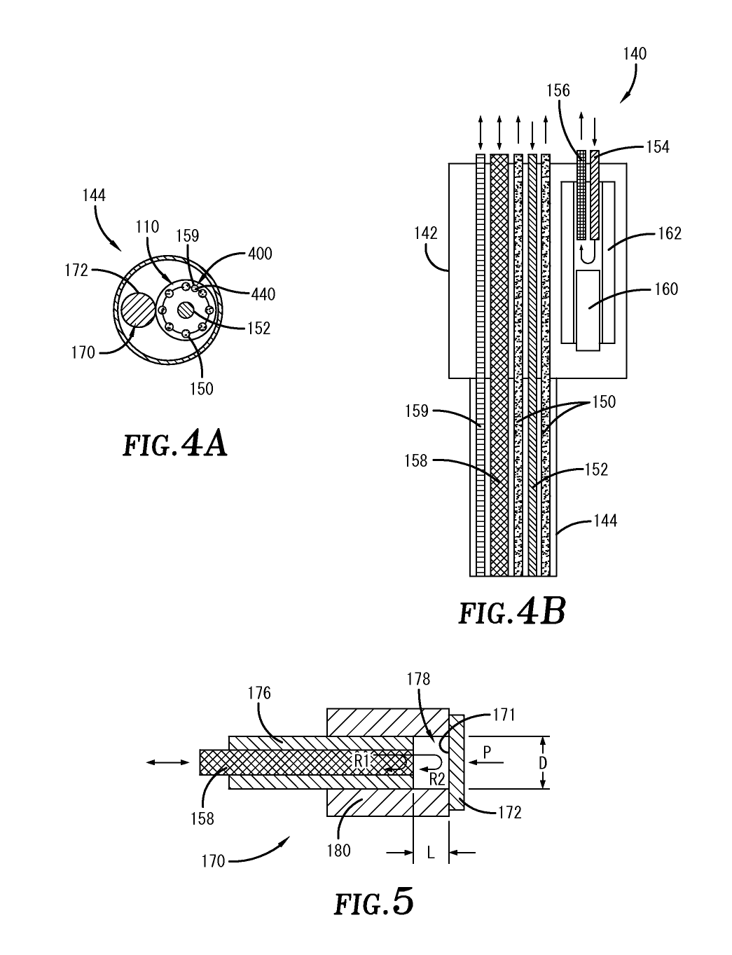

The DRS-sensing channel 110 may comprise a channel, wherein light from the first light source 106 illuminates the biological tissue sample, or specimen 146, whereupon at least one detection fiber 150 is able to capture the reflected light (i.e., spectral data), which is ultimately provided to the spectrometer 104 via the fiber array shown in cross-sectional views of FIGS. 3A and 4A. The cross-sectional views 3A and 4A of the fiber array are associated with the open-ended terminus of probe tip 144, which is configured to be brought into contact with the biological tissue specimen 146 during use of the sensor system 100. Notably, each individual detection fiber 150 may run the entire length of collection leg 124 and the main portion of probe 138.

The calibration channel 120 includes an illumination fiber leg 126 and a calibration return leg 128, as shown in FIG. 1. The calibration return leg 128 is configured to be coupled to the second spectrometer 108 at coupler 118. The illumination fiber leg 126 is configured to share the first light source 106, such as a white LED, with the sensing channel 110, and can collect a calibration spectrum concurrently with the tissue measurement, which can be used for real-time instrument and probe calibration. The illumination fiber leg 126 may comprise two source fibers, such as an illumination source fiber 152 that is utilized with the sensing channel 110 for illuminating the specimen, and a calibration source fiber 154 that is utilized in calibration channel 120 for internal calibration, as shown in FIG. 2B. The illumination source fiber 152 and the calibration source fiber 154, respectively, may run parallel to each other within the illumination fiber leg 126. Illumination source fiber 152 may extend into the rigid probe tip 144 for contact and sensing of the specimen tissue 146. In one embodiment, each of the illumination source fiber 152 and the calibration source fiber 154 may comprise the same diameter fiber or fibers. Furthermore, all of the fibers may be made from the same materials (e.g., same fiber clad, core, etc.), and may comprise the same numerical aperture (NA) for identical bending response. The calibration source fiber 154 is discussed below with respect to FIG. 3B, but may not extend all the way to specimen surface 146. Calibration return leg 128 of calibration channel 120 is also discussed with respect to FIG. 3B and can be useful for collecting calibration light generated at first light source 106 and reflected by a reflective material 160 (FIG. 3B) and transmit the reflected light to spectrometer 108. In one embodiment, smart fiber-optic sensor 105 may be used to concurrently collect and measure the spectral data of the first light source 106 and the spectral data of the tissue specimen 146. Such configuration is advantageous because it can account for real-time light source intensity fluctuations and fiber bending loss (i.e., light intensity fluctuations caused by bending the instrument). For example, the effect of bending on the illumination source fiber 152 is assumed to be the same as that of calibration source fiber 154. Also, light source warm-up and separate calibration measurements are also unnecessary with the use of the smart fiber-optic sensor system.

In addition, the fiber-optic pressure sensor channel 130 may comprise the second illumination fiber leg 135 and a pressure return leg 133. The second illumination fiber leg 135 is coupled to the second light source 115 at a second light source coupler 125, while the pressure return leg 133 is coupled to the spectrometer B 112 at a spectrometer coupler 122. The second illumination fiber leg 135 and the pressure return leg 133 may comprise a single lead in/out fiber (single-mode or multi-mode) forming a low-coherence DFPI at the end face of the lead in/out fiber proximate to the probe tip 144. The second light source 115 may comprise an 850 nm light emitting diode (LED) with a spectral width of about 30 or 60 nm, such as that provided by LEDs manufactured by Appointech Inc.

In order to monitor the physical changes of the specimen tissue 146 that occur due to the application of heat from the electrosurgical device used during robot-assisted laparoscopic procedures, the temperature sensor channel 131, as shown in FIG. 1 is used. The fiber-optic temperature sensor channel 131 may comprise the second illumination fiber leg 135 and a temperature return leg 136. The temperature return leg 136 is coupled to the spectrometer D 132 at a spectrometer coupler 137. The second illumination fiber leg 135 and the temperature return leg 136 comprise a single lead in/out collection optical-fiber (i.e. single-mode) that forms an interferometric temperature sensor that traverses the temperature return leg 136, and that detects temperature of that specimen 146 at the probe tip 144. As previously discussed, the second light source 115 may comprise an 850 nm LED, as well as any other suitable light source.

Thus, as previously discussed, the smart fiber-optic sensor 105 includes a collection fiber leg 124, a first illumination fiber leg 126, a second illumination fiber leg 135, a calibration return leg 128, a pressure return leg 133, and a temperature return leg 136. The smart fiber-optic sensor 105 also includes a probe tip portion 140, which is coupled to a breakout tube 136 by a probe leg 138. In particular, the probe portion includes the probe tip 144. In one aspect, the probe tip portion 140 may include a calibration housing portion 142 and the rigid probe tip 144. The probe tip 144 is configured to contact the surface of the biological tissue specimen 148, such that the optical fibers of the DRS channel 110, the pressure channel 130, and the temperature channel 131 are brought into proximity or contact with the tissue specimen 146 as necessary to carry out the functions discussed herein. In a further aspect, the tissue specimen 146 may comprise a tissue sample or any turbid medium. Thus, the contact pressure at the probe tip/specimen interface may be calculated and controlled by the robotic surgical system 147 using the fiber-optic pressure-sensing channel 130. Additionally, the contact temperature at the probe tip/specimen interface may also be calculated and manually controlled by the user, or automatically controlled by the robotic surgical system using the fiber-optic temperature-sensing channel 131.

FIG. 2 illustrates various light source and collection fibers for use in the smart fiber-optic sensor system 100. The light source and collection fibers traverse various legs of the smart fiber-optic system 100 previously described in FIG. 1. In one embodiment, the DRS sensing channel 110 comprises at least one illumination source fiber 152 that traverses the illumination fiber leg 126 and at least one collection fiber 150 that traverses the collection leg 124. Each of the illumination source fiber 152 and the collection fiber 150 traverse the entire length of the probe leg 138 and extend through probe tip 144 and are polished flush with the end of probe tip 144. FIG. 2 also shows the collection fiber 150, which connects to the spectrometer 104 at coupler 114. The calibration channel 120 comprises at least one calibration source fiber 154 and at least one calibration return fiber 156. FIG. 2 also shows the illumination source fiber 152 of the sensing channel 110 and the calibration source fiber 154 of the calibration channel 120 each of which traverse the illumination fiber leg 126 and connect to first light source 106 at coupler 116. The calibration source fiber 154 traverses the probe leg 138 and loops back or terminates in the calibration housing portion 142 of the probe tip portion 140. In one aspect, the illumination source fiber 152 comprises a 400/400/480 μm fiber for illumination. The calibration source fiber 154 may comprise another fiber of the same diameter for use in calibration. The illumination source fiber 152 may provide a sensing depth sufficient to probe both the epithelium and the stroma within the biological tissue specimen 146. These dimensions are not limiting as any suitable fiber diameter can be used for the illumination source fiber 152 and/or the calibration source fiber 152. It is also contemplated that the fibers may be suitably sized and/or shaped to comprise a variable sensing depth by using either a multi-separation probe or angled probe design. In one aspect, the illumination fiber leg 126 may comprise any suitable number of calibration source fibers 154 and illumination source fibers 152, and is not limited to the size/shape/or quantity illustrated.

FIG. 2 also shows at least one calibration return fiber 156 that carries reflected light that traverses the calibration return leg 128 and the probe leg 138 to ultimately connect to the spectrometer 108 at coupler 118. Together, the calibration source fiber 154 and calibration return fiber 156 form the calibration channel 120. FIG. 2 also shows a lead in/out fiber 158 that comprises a pressure sensor fiber that traverses each of the second illumination fiber leg 135, the pressure return leg 132, and the probe leg 138. In one embodiment, the pressure sensor channel 130 comprises the second illumination fiber leg 135 and the pressure return leg 132, wherein the lead in/out fiber 158 is disposed in each leg. The various legs may comprise any suitable material for housing one or more fiber-optics. For example, the legs may comprise stainless steel, or metallic tubes, which include bored holes for placement of fiber-optic fibers. In another embodiment, the legs may comprise hollow tubes where a filling material is injected or disposed, which can surround the respective optical fibers.

FIG. 2 also shows that the temperature sensor channel 131 includes the second illumination fiber leg 135 and the temperature return leg 136, wherein a lead in/out fiber 159 is disposed in each leg. Thus, the temperature fiber 159 traverses the second illumination fiber leg 135, the temperature return leg 136, and the probe leg 138. As such, light delivered from the illumination fiber leg 135 to the tissue 146 is reflected by the tissue 146, and is carried by through the temperature return fiber 159 via the temperature return fiber 159 for receipt at the spectrometer D 132 via coupler 137. Together the illumination fiber leg 135 and the temperature return leg 136 form the temperature channel 131.

FIGS. 3A-B and 4A-B illustrate various cross-sectional views of the probe tip 144 and calibration housing portion 142. In one aspect, the probe tip 144 may comprise ridged elements (not shown) to provide stability and/or interfacing capability for the smart fiber-optic sensor 105. FIG. 3A illustrates one embodiment of a cross-sectional view of the probe tip 144 as it appears from a distal end for contacting the tissue specimen 146. As such, the probe tip 144, wherein the illumination source fiber 152 and the collection fiber 150 are disposed having longitudinal axes that are parallel to a longitudinal axis of the fiber-optic pressure sensor 170, as shown in FIG. 5, and to the longitudinal axis of the fiber-optic temperature sensor 159. As shown in FIG. 3A, the illumination source fiber 152, the collection fiber 150, the pressure sensor diaphragm 172 of the pressure sensor 170, and the terminal end of the temperature sensor formed by the in/out fiber 159 are polished to the same plane, such that each are flush with the terminal end of the probe tip 144 so that they can be brought into physical contact with the surface of the target specimen 146 during spectroscopic measurement using the robotic surgical system 147. The probe tip 144 may comprise a stainless-steel tube or ferrule 157, which surrounds illumination source fiber 152, the DRS collection fiber 150, the pressure-sensing in/out fiber 158 of the pressure sensor 170, and the temperature sensing fiber 159 of the temperature sensor.

FIG. 3B illustrates an exemplary smart fiber-optic sensor tip portion 140 that includes the calibration housing 142 for the self-calibration optical fibers and the probe tip 144. In one embodiment, the probe tip portion 140 receives both the illumination source fiber 152 and the calibration source fiber 154 from the illumination fiber leg 126. Specifically, the illumination source fiber 152 passes completely through housing section 142 in order to interface with the specimen 146, however the calibration source fiber 154 terminates within housing section 142. In one embodiment, light exits the calibration source fiber 154 and is directed so that it is incident on reflective material 160. The reflective material 160 may include a mirror, a polished metal element (e.g., a polished metal wire), a reflective rod, and/or any other suitable reflective material, in one aspect, the reflective material 160 may comprise any shape and/or material. In one embodiment, the calibration source fiber 154 and the return fiber 156 may be inserted into a sealed tube that is filled with a material having diffusely reflective capabilities, such as Spectralon® reflective material manufactured by Labsphere. After being incident upon the reflective material 160, the light may be reflected towards a calibration return fiber 156, which receives the light and carries it to the spectrometer, or spectrometer channel 108. A flexible, stainless-steel tube or ferrule 162 may surround the calibration source fiber 154 and the calibration return fiber 156.

In one embodiment, the calibration source fiber 154 and the calibration return fiber 156 may comprise the same fiber (i.e., a source/return calibration fiber). For example, a single source-return calibration fiber may originate from the light source 106, enter the housing section 142, and then bend or loop back in such a manner that the calibration source/return fiber exits the housing section 142. That is, the calibration source-return fiber can be bent within the housing section 142 in the smart fiber-optic sensor, such that the calibration source fiber 154 functions as the calibration return fiber (since a mirror or other reflective element is not used). The calibration fiber would then be configured to interface with spectrometer channel 108 via the calibration return leg 128. In particular, the reflective material 160 would not be utilized in this particular embodiment. Furthermore, although the cross-sectional view of FIG. 3B shows one calibration return fiber 156, additional calibration return fibers may be used. For example, additional calibration return fibers 156 may be implemented as backup return fibers in case the primary return fiber fails or if additional calibration channels are to be implemented.

In another embodiment, the smart fiber-optic sensor 105 may comprise a calibration channel that can be used to record the lamp spectrum and instrument/fiber responses concurrently with tissue measurements. For example, at least one calibration source fiber 154 can transmit, or communicate calibration light and calibration return fiber 156 can collect, or communicate the calibration light reflected by the reflective material 160 within calibration housing 142 and transmit it to the spectrometer 108. The calibration spectra from the calibration channel can be detected by the spectrometer 108 and can be used for the calibration of the specimen spectrum that is obtained concurrently.

In a further embodiment, the calibration source fiber 154 of the calibration channel 120 may have the same diameter as, and run along the illumination fiber 152 of the sensing channel 110. The calibration return fiber 156 may be the same diameter as the collection fiber 150 in the tissue channel to achieve an identical bending response. To account for wavelength dependence, a correlation factor may be applied before being processed with the spectral data collected from the tissue specimen 146. For example, because the calibration channel 120 may have wavelength responses that differ from the wavelength responses exhibited in the sensing channel 110, the wavelength response in the calibration channel 120 may require correction and/or compensation. To correct the wavelength dependence of the calibration channel 120, a spectral measurement may be taken from a reflectance standard (e.g., a Spectralon puck), which is characterized by a flat wavelength response. A correction factor may then be generated for each sensor or probe by dividing the spectral data of the reflectance standard by the self-calibration spectrum concurrently obtained with spectral data of the reflectance standard. For example, the correlation factor may be characterized by the following equation:

F_{corr}(λ)=Puck(λ)/SC_{puck}(λ),

where Puck (A) is measured from a Spectralon puck by the sensing channel 110, and SC_{puck} (A) is the concurrent spectrum measured by the calibration channel 120. The calibrated reference phantom and tissue spectra can be input into the fast-scalable Monte Carlo inverse model, which extracts the tissue μs′ and μs values from which tissue absorber concentrations can be derived. With the smart fiber-optic sensor system 100, no separate calibration measurements are needed. Exemplary Monte Carlo algorithms suitable for use with the subject matter described herein can be found, for example, in international patent application number PCT/US2007/006624 to Palmer et al.; international patent application number PCT/US2008/0270091 to Ramanujam et al.; and U.S. Pat. No. 7,835,786 to Palmer et al., the entireties of which are incorporated herein by reference, in an alternative embodiment, a diffusion algorithm or inverse diffusion algorithm may be used instead of a Monte Carlo algorithm. Notably, tissue measurements can be started right after the instrument is turned on and fiber bending loss can be accounted for in real time. As a result, such features may provide substantial time savings.

FIGS. 4A-B illustrate another cross-sectional embodiment of the probe tip 144, which could be used as an alternative to that shown in FIGS. 3A-B. If used, the probe tip 144 shown in the cross-section of FIG. 3B would adjust accordingly to account for the arrangement of one or more detection or collection fibers 150 about the source fiber 152, as illustrated in FIG. 4B. FIG. 4A illustrates the illumination source fiber 152 that is coupled to the light source 106 substantially disposed in the center of eight collection fibers 150, and the temperature sensor 159 to collect the diffusely-reflected light from the specimen 146 at one or more wavelengths and to detect the temperature of the specimen 146. In one embodiment, the eight detection fibers 150 may be 200 μm in diameter. However, in other embodiments the multiple detection fibers 150 may comprise a plurality of illumination fibers, while the single illumination fiber 152 may comprise one or more collection fibers. That is, the collection fiber 150 may comprise a single optical fiber that is surrounded by one or more illumination fibers 152. However, it should be appreciated that any desired number and configuration of the illumination fibers and any desired number and configuration of the detection fibers may be used by the sensor system 100. In addition, the probe tip 144 may be 9.3 cm long, having a diameter of 2.1 mm, and can fit within the lumen of a 14-gage biopsy needle cannula; however, any other suitable dimension may be used. For illustration purposes, the embodiments of the cross-sectional view of the probe tip 144 are shown by FIGS. 3 and 4; however, any other suitable arrangement and/or number of source and collection fibers, as well as temperature sensor may be used by the present invention. For example, the illumination core may include a plurality of illumination fibers (i.e. instead of one single illumination source fiber 152 to obtain an illumination core diameter that maximizes both the coupling efficiency for the light source and the signal-to-noise ratio (SNR) for fluorescence measurements (if applicable). Furthermore, instead of using eight defection fibers 150, any number of detection fibers may be used, such as one detection fiber. For example, the one detection fiber 150 may be surrounded by four illumination source fibers 152. In another embodiment, the illumination source fiber 152 can be used to emit light on the tissue specimen 146 being examined. Light can be generated by first light source 106 and provided directly to the illumination fiber leg 126 of the smart fiber-optic sensor 105 or via a monochromator (not shown). Notably, light carried by the illumination source fiber 152 and the calibration source fiber 154 can be characterized by the same spectral data. It should be appreciated that the illumination source may compirse any number of optical fibers.

FIG. 5 shows a fiber-optic pressure sensor 170, which comprises a diaphragm-based Fabry-Perot interferometric (DFPI) sensor that is formed at a cleaved end face of lead in/out fiber 158. Details regarding DFPI sensor interrogation can be found in references [10] and [11], the entireties of which are incorporated by reference herein. The pressure sensor 170 may be illuminated by an 850 nm (or any other near infrared (NIR) wavelengths) light emitting diode (LED) with a spectral width over 30 or 60 nm. The 850 nm LED can be turned on during all spectroscopic measurements, and the spectral output of spectrometer channel 115 can be processed immediately to calculate the pressure at the probe-tissue interface. The lead in/out fiber 158 may be disposed within the ferrule 176 and tube 180. In one embodiment, the ferrule 176 and tube 180 may be formed of fused silica, although any suitable material may be used. High-temperature epoxy or a high-power laser may be used to bond the lead in/out fiber 158, the ferrule 176, the tube 178, and the diaphragm 172 together. A static or dynamic external pressure P is applied on the outer side of the diaphragm 172 during contact of the probe 144 on the specimen 146, so as to deflect the diaphragm 172 towards the tip of the fiber 158, reducing the length L of an air cavity 178. The extremely low thermal expansion coefficient of fused silica makes the air cavity 178 length L highly insensitive to environmental temperature change. The outer diameter D of the pressure sensor 170 may comprise a dimension in the range from about 3 to 5 mm. The length L of the air cavity 178 may be measured using low-coherence interferometry. In general, light from the second light source 115 may be launched into the sensor head using the lead in/out fiber 158. The low-coherence light propagates into air cavity 178, where the light beam is partially reflected by the end face of the in/out fiber 158, as illustrated by R1, and at least partially reflected at an inner surface 171 of the diaphragm 172, as illustrated by R2. The reflected light beams R1 and R2 then propagate back to the detector or spectrometer 112 through the lead in/out fiber 158 and interfere with each other. By analyzing the interferogram that is generated, the length L of the air cavity 178 can be calculated in real time with nanometer-to-sub-nanometer accuracy. Thus, the application of external pressure P (e.g., contact pressure at sensor-specimen interface) on the outer surface of the tissue specimen 146 bends the diaphragm 172 toward the fiber 158 and thus, changes the length L of air cavity 178. The applied pressure or force can be calculated by measuring the change of the cavity length L from atmospheric pressure. The reflected light beams R1 and R2 may comprise reflectance data and/or pressure sensor spectral data. It should be appreciated that the pressure sensor 170 may be operated within a linear region (i.e. a small region on one side of an interference fringe near its quadrature point) for optimal sensitivity and the largest signal bandwidth, which is required for detection of dynamic pressure waves. It should be appreciated that DFPI sensors, such as pressure sensor 170 may attain a sensitivity of 87 mV/psi and a high resolution of 0.023 psi (or 159 Pa) and a dynamic range over 100 psi [11] [12].

Still referring to FIG. 5, the pressure sensor 170 may be fabricated in one embodiment by inserting a 50/125 μm multimode fiber into a fused silica ferrule having an outer diameter of 1.5 mm and inner diameter of 127 μm. The fiber may be bonded to the ferrule using high-temperature epoxy, and the tip of the fiber can be polished down to the level of the ferrule with optical quality. A 100 μm thick fused silica diaphragm may be bonded to the polished end face of a 1.0 cm long fused silica outer tube with a 1.5 mm inner diameter. The ferrule with fiber may be inserted into the outer tube at the end without the diaphragm until a cavity length L of about 10-14 μm can measured by an interrogation instrument. The ferrule and the outer tube may be permanently bonded together with high-temperature epoxy, and the air cavity 178 length L is then monitored and adjusted during a curing process. The diaphragm can be polished, along with the probe tip 144 down to about 50 μm in thickness.

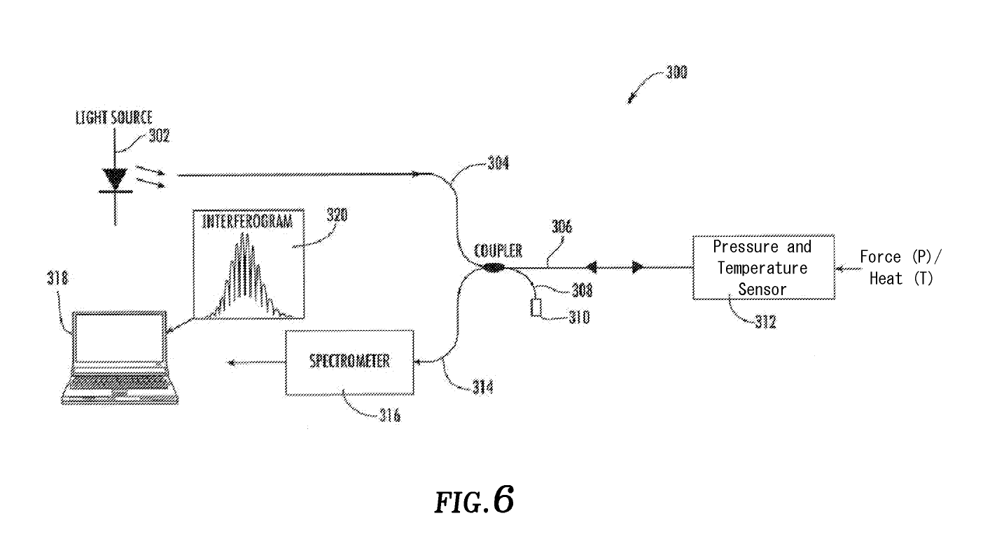

FIG. 6 shows a block diagram of an embodiment of the pressure-sensing channel, generally designated 300. Pressure-sensing channel 300 includes components essentially corresponding to pressure-sensing channel 130 of FIG. 1. The real-time pressure detected by the probe can be displayed on a processing unit, such as a portable laptop computer 318, so that an operator can adjust the force applied to the probe. Alternatively, the increased temperature value may be coupled to an automated RAL. Programming can be used such that only if the desired pressure is reached during a scan, will the specimen spectra be saved and processed. A light source 302 can generate a low-coherence light source. The light source 302 may comprise an 850 nm LED with 30 or 60 nm spectral widths and traverse an illumination leg 304. A multimode fiber coupler can be used to couple a sensor head 312 to light source 302 and sensor head 312 to spectrometer 316. For example a pressure-sensing leg 306 can be coupled at coupler to send a signal to spectrometer 316 over return leg 314 upon receiving and reflecting low-coherence light from light source 302. An inactive portion of sensing leg 308 can be placed in an index matched terminal 310 as to not interfere with the light and/or signal of pressure-sensing leg 306. Sensor head 312 can comprise pressure sensor 170 discussed above and can optionally comprise a DFPI sensor head. A probe pressure range of 0-15 psi can be selected. For best measurement consistency, a pressure resolution of 0.1 psi can be selected. The sensitivity of the DFPI sensor under an external force or contact pressure difference δ can be expressed as:

δ=y₀(P)/P=1.74×10⁻⁵a⁴/h³(nm/psi)

where y₀ (nm) is the deflection of the diaphragm at the center, a=D/2 (μm) is the effective radius of diaphragm 172 (FIG. 5) and h (μm) is the thickness of diaphragm 172. The sensitivity as a function of the diaphragm thickness at three diaphragm diameters (D=1.0, 1.5 and 2.0 mm) is plotted in FIG. 8. An interferogram 320 of signal sent over return leg 314 can be analyzed using a simple fringe peak tracking algorithm or function, in one embodiment, a measured cavity length of a DFPI sensor showed a ±0.5 nm stability over 48 hours with a dynamic range over ten microns and ±1.5-nm shift in the temperature range of 10 to 45° C. When a diaphragm of 1.5 mm effective diameter and a thickness of 50 μm can be selected, a sensitivity of 44 nm/psi, and thus a pressure resolution of 0.068 psi can be easily achieved within 10-45° C.

A non-transitory computer readable medium comprising computer executable instructions, that when executed by a processor of a computer 318, can perform steps comprising collecting spectra from the specimen, calculating and displaying probe pressure for the operator to manually adjust it, adjusting integration time, as necessary, calibrating the sample spectrum, perform spectral analysis, and display the extracted specimen optical properties and physiological parameters, in one aspect, data can be automatically stored if and only if pressure is within an optimal range and upon successful calibration. An optimal range can comprise a minimal optimal range wherein spectral data will be minimally affected by the contact pressure, it is anticipated that an optimal range will comprise 0 to 20 or 0 to 30 psi. The optimal range can comprise a preset value for a processing unit to base decisions off of. For example, a processing unit can automatically analyze and save specimen spectral data if contact pressure is within the minimal optimal range. The minimal optimal range may be dependent on the type of tissue analyzed (e.g., cervical, oral) and the underlying tissue composition. Computer executable instructions can control the smart fiber-optic system, load reference phantom and default parameters (such as integration time and desired pressure range), collect spectra from specimen and pressure sensor, calculate and display the probe pressure for the operator to manually adjust it, adjust integration time, calibrate the sample spectrum, perform spectral analysis, and display the extracted tissue optical properties and physiological parameters. It is expected that the time required to measure and analyze the spectra from a specimen be less than two seconds.

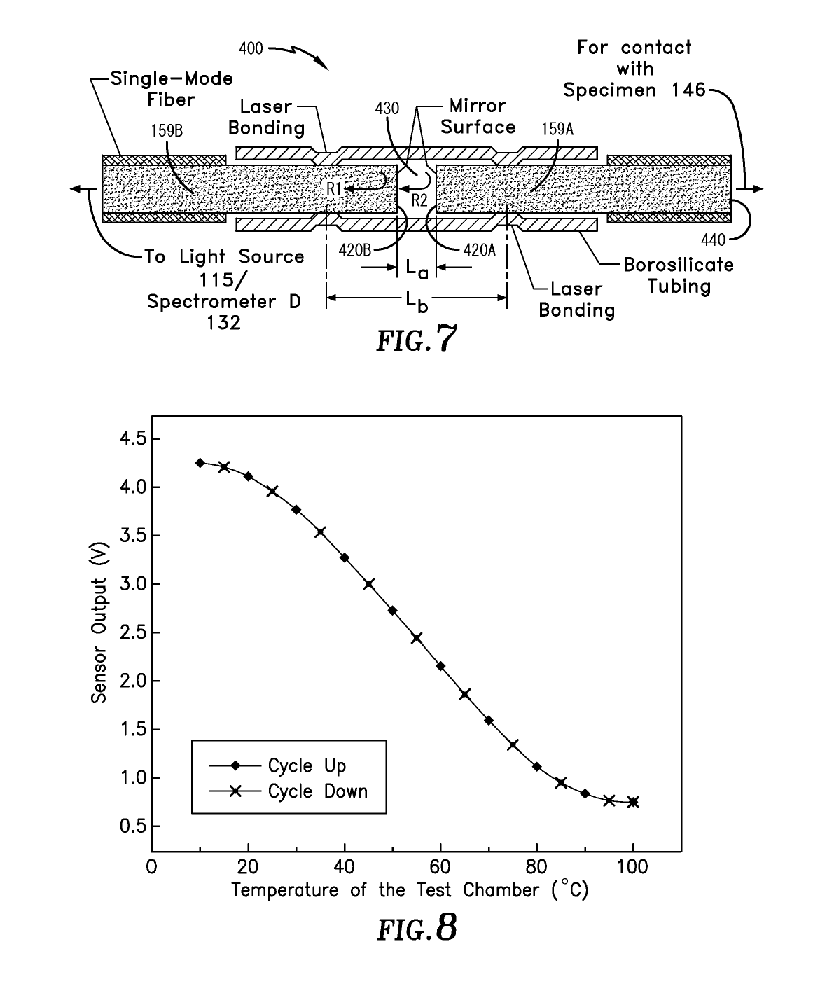

in one aspect, FIG. 7 shows an interferometric temperature sensor 400 that is formed at the cleaved end face of the lead in/out temperature return fiber 159, as shown in FIG. 7. The sensor 400 is configured so that the return fiber 159 is formed by a pair of fiber-optic legs 159A and 159B, which are formed of fused silica. The fiber optic legs 159A-B are joined by an attachment tube 410. The fiber-optic legs 159A and 159B comprise single-mode fibers that form a low-finesse Fabry-Perot interferometer inside the capillary or attachment tube 410. It should be appreciated that the attachment tube 410 may comprise a diameter of about 380 um; however, any other suitable diameter may be used. In addition, the faces of the ends 420A and 420B of respective legs 159A and 159B are polished to have a mirrored surface, but is not required. As such, leg 159B is attached at one end to the spectrometer D 132 and the second light source 115 and at another end 420B to the tube 410, while leg 159A is attached at one end 420A to the tube 410 and at another end 440 to the probe tip 144, where the exposed end 440 of the leg 159A that is positioned proximate to the probe tip 144 is configured to be placed into contact with the tissue specimen 146 during use of the sensor system 100. As such, the ends 420A and 420B of each of the fiber-optic legs 159A and 159B are received within and attached to the attachment tube 410, such that the ends 420A and 420B are spaced apart from each other by an initial predetermined distance to form a cavity 430, which has a cavity length (Lₐ). in one aspect, the ends 420A-B of the legs 159A-B may be attached to the tube 410 via laser bonding or any other suitable means of attachment. In another aspect, the tube 410 may be formed of borosilicate or any other suitable material. It should be appreciated that the initial cavity length Lₐ may be identified using the same interrogation system used by the pressure sensing channel 300 discussed herein with regard to FIG. 6, using spectrometer D 132 and the interferogram processing algorithm for the pressure sensor previously discussed.

In particular, the measurement of temperature T of the tissue specimen 146 using the sensor 400 is based on the following equation: Lₐ′=Lₐ+[(a_{b}−a_{f})·L_{b}]·(T−T₀), where Lₐ′ is the initial cavity length, L_{b} is the gauge length of the tube 410 at room temperature T₀, α_{b} is the coefficient of thermal expansion (CTE) of the borosilicate tube 410, and α_{f} is the coefficient of thermal expansion (CTW) of the fused silica fiber of fiber-optic legs 159A and 159B. Furthermore, it is assumed that L_{b}>>Lₐ.

During testing of the temperature sensor 400 inside a temperature chamber, the results shown in FIG. 8 were attained, whereby an accuracy around +/−0.1° C. was achieved within a temperature range of between about 10-100° C.

During operation of the temperature sensor 400, the exposed end 440 of the temperature sensor 400 is brought into contact with the tissue specimen 146. Next, light from the second light source 115 is launched into the temperature sensor 400 via leg 159A using the lead in/out fiber 159 to which the second light source 115 is coupled. The low-coherence light propagates into the air cavity 430, where the light beam is partially reflected by the end 420B of the leg 159B, as shown by R1, and at least partially reflected by the surface of the end 420A of the fiber leg 159A, as shown by R2. The reflected light beams R1 and R2 then propagate back through the in/out fiber 159 to the spectrometer D 132, where the reflected light beams interfere with each other. Thus, by analyzing the interferogram that is generated, the length Lₐ of the cavity 430 can be calculated in real-time with nanometer to sub-nanometer accuracy. Thus, the temperature applied at the exposed end 440 of the sensor 400 from the tissue specimen 146 changes the cavity length Lₐ due to the thermal expansion or contraction of the tube 410. The reflected light beams R1 and R2 may comprise reflectance data and/or temperature sensor spectral data. The temperature sensor 400 may be operated within a linear region (i.e. a small region on one side of an interference fringe near its quadrature point) for optimal sensitivity and the largest signal bandwidth. It should be appreciated that the temperature sensor 400 may have an operating range from about room temperature to about 200° C.

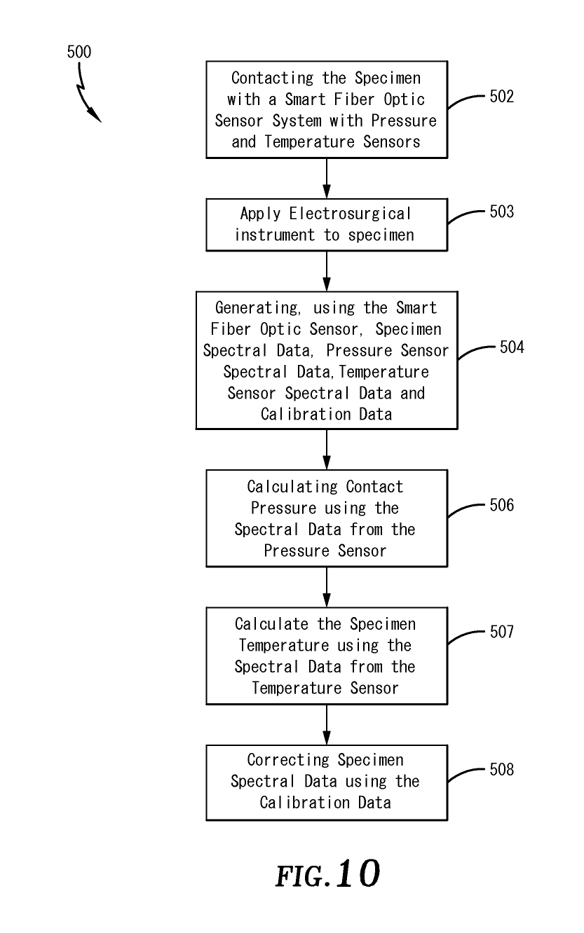

FIG. 10 is a flow diagram for an exemplary process 500 for using the smart fiber-optic pressure sensor system in association with a robotic surgical system 147, as described herein. Initially, step 502 includes contacting the specimen 146 with the probe tip 144 of the smart fiber-optic sensor system 100 by moving the robotic end-effector 148 to the desired position. The pressure at which the smart fiber-optic sensor contacts the specimen, that is, the contact pressure at a probe-specimen interface may be controlled using the smart fiber-optic sensors and methods described herein, by calculating contract pressure from spectral data collected from the pressure sensor. For example, the robotic surgical system may by configured with the necessary control systems to process the acquired pressure data and control the position the end-effector 148 so that the pressure detected at the probe tip 144 is within a desired range, or may be manually controlled by the operation of the robotic surgical system. Next, at step 503, an electrosurgical cutting instrument or ultrasound cutting instrument is used to cut or otherwise manipulate or treat the specimen tissue 146. Next, at step 504, the smart fiber-optic sensor system 100 generates specimen spectral data, pressure sensor spectral data, temperature spectral data and calibration spectral data.

Generating specimen spectral data may comprise transmitting a illumination light via at least one illumination fiber within the sensing channel from a first light source to the specimen 146 and collecting the specimen spectral data using at least one detection fiber in the sensing channel, wherein the specimen spectral data can comprises the illumination light from the first light source diffusely reflected from the specimen at one or more wavelengths.

Step 506 includes calculating contact pressure at the probe tip 144 using the spectral data from the pressure sensor. The pressure sensor spectral data may comprise reflectance data reflected along a cavity length of the DFPI, as discussed above. The pressure sensor may be disposed at the probe tip 144 of the smart fiber-optic sensor 100. The pressure is calculated by transmitting a low-coherence illumination light using a fiber-optic fiber to a DFPI pressure sensor and collecting spectral data reflected by the pressure sensor via the same fiber-optic fiber. As illustrated in FIG. 6, low-coherence light propagates through the coupler to reach a lead in/out fiber. The output light from the DFPI propagates through the coupler and a signal (from R1 and R2, FIG. 5) can be detected by the spectrometer that comprises an interferogram from which the cavity length L is calculated using an algorithm. The cavity length L may range from a few to several hundred micrometers. External pressure or pressure resulting from the contact of the probe tip 144 with the tissue specimen 146 comprises results in physical contact with an outer surface of the diaphragm, thereby bending the diaphragm toward the fiber, thus changing the length L of the air cavity. The applied pressure, or force, can therefore, be calculated by measuring the change of the cavity length from atmospheric pressure. A determination can be made regarding whether to analyze and save the spectral data from the sensing and calibration channels based on the pressure data. If pressure is not from a minimal preset range, the contact pressure may be too great thereby affecting specimen spectral data, thus, resulting in error and potentially inaccurate specimen data. Contact pressure at the probe/specimen interface can be adjusted and/or controlled such that the measured pressure stays within a specified, preset pressure range. In one aspect, the contact pressure may be controlled by an operator or technician based on real time pressure readings, or may be automatically controlled by the robotic surgical system 147. As noted earlier, the first light source shared by sensing and calibration channels comprises a first emission spectra and the second light source utilized by the pressure-sensing channel comprises a second emission spectra, and the first and second emission spectra do not overlap.

Step 507 includes measuring the temperature T of the specimen tissue 146 as detected by the temperature sensor 400, as previously discussed. That is, the change in cavity length Lₐ due to the change in temperature of the capillary tube 410 is detected using the interferogram that is generated by the reflected light beams R1 and R2 and using the algorithm discussed above. Once the temperature T is identified, a user of the robot-assisted surgical system 147 may reduce the intensity of the electrical current applied by the electrosurgical cutting device at the specimen tissue 146 in order to reduce the temperature of the tissue 146, and prevent damage thereto. In addition, the robotic surgical system 147 may be configured to automatically take corrective action to control the electrosurgical device to prevent overheating and damage to the tissue 146. It should be appreciated that this step can be performed at any desired step in process 500.

Next, step 508 includes correcting the specimen spectral data by using the calibration data. Correcting specimen spectral data may comprise transmitting calibration light via at least one calibration source fiber disposed in calibration channel, wherein the calibration light and the illumination light of the sensing channel can be generated simultaneously from the shared, first light source. Correcting specimen spectral data may further comprise collecting correcting spectral data associated with the calibration light via at least one calibration return fiber of the calibration channel contemporaneously with the collection of the spectral data of the specimen. The specimen spectral data received from the sensing channel may be corrected using the calibration spectral data received from the calibration channel.

Optional steps that may be incorporated in the process 500 include analyzing and storing the specimen spectral data. Analyzing and storing the collected spectral data may comprise analyzing the calibrated spectral data to extract the specimen optical and physiological properties of the tissue specimen 146 using, for example, an inverse MC model for reflectance. In one aspect, the data can be automatically analyzed and stored using a determination based upon the pressure data. If the pressure data is within a specified, preset range, then the specimen spectral data and calibration data can be automatically analyzed and/or stored.

Thus, it is desirable to utilize a powered smart sensor system for performing in vivo quantitative DRS of soft tissues at a wavelength range from about 420-720 nm. The applications related to cancer screening in a global population can be greatly improved by the methods and systems disclosed herein. The smart sensor technology disclosed herein can incorporate innovations to several component areas. For example, white LEDs, miniature spectrometers, and a smart fiber-optic sensor can reduce the complexity, size, and cost of conventional optical spectroscopy systems. The systems and methods disclosed herein also minimize the amount of technical skill required to perform optical spectroscopy for early cancer detection applications. The compact integration of a tissue sensing portion, a pressure sensor, and a calibration portion into a single fiber-optic probe enables significant improvement in accuracy and robustness for extraction of tissue optical properties. By limiting or controlling the probe pressure and performing real-time calibration both systematic and random errors in reflectance measurements can be reduced. In addition, by providing temperature sensing of the tissue prevents tissue damage in the form of charring and burning. Further, the sensitivity and specificity for early cancer diagnosis can be improved.

Although the smart sensor discussed herein can be useful for screening and diagnostics for cancers such as oral and cervical cancers, it is not limited thereto. The systems and methods disclosed can be translated to any organ or tissue site, such as the skin, bladder, etc. and can also be used for non-cancer applications such as monitoring vital signs during major surgeries in an intra-operative setting. The systems and methods disclosed herein can be used for any optical spectroscopy application.

Therefore, one advantage of the present invention is that a smart fiber-optic sensor for optical spectroscopy includes a temperature sensor that allows surgeons to identify when the temperature of a tissue specimen being treated by an electrosurgical cutting device exceeds a desired level to prevent unwanted charring and damage to the tissue at the surgical site. Another advantage of the present invention is that a smart fiber-optic sensor for optical spectroscopy includes a temperature sensor that allows surgeons to identify when the spread of heat from an electrosurgical device, such as a laparoscopic surgical device, exceeds a desired area. Still another advantage of the present invention is that a smart fiber-optic sensor for optical spectroscopy is configured for attachment or made integral with an articulating end-effector of a robotic surgical system, such as a robot-assisted laparoscopic (RAL) surgical system.

Thus, it can be seen that the objects of the invention have been satisfied by the structure and its method for use presented above. While in accordance with the Patent Statutes, only the best mode and preferred embodiment has been presented and described in detail, it is to be understood that the invention is not limited thereto or thereby. Accordingly, for an appreciation of the true scope and breadth of the invention, reference should be made to the following claims.

### REFERENCES

The disclosure of each of the following references is hereby incorporated herein by reference in its entirety.

[1] Schwarz, R. A., W. Gao, D. Daye, M. D. Williams, R. Richards-Kortum, and A. M. Gillenwater, Autofluorescence and diffuse reflectance spectroscopy of oral epithelial tissue using a depth-sensitive fiber-optic probe. Appl Opt, 2008. 47(6): p. 825-34.

[2] Wang, A., V. Nammalavar, and R. Drezek, Targeting spectral signatures of progressively dysplastic stratified epithelia using angularly variable fiber geometry in reflectance Monte Carlo simulations. J Biomed Opt, 2007. 12(4): p. 044012.

[3] Liu, Q. and N. Ramanujam, Sequential estimation of optical properties of a two-layered epithelial tissue model from depth-resolved ultraviolet-visible diffuse reflectance spectra. Appl Opt, 2006. 45(19): p. 4776-90.

[4] Utzinger, U. and R. R. Richards-Kortum, Fiber optic probes for biomedical optical spectroscopy. J Biomed Opt, 2003. 8(1): p. 121-47.

[5] Reif, R., M. S. Amorosino, K. W. Calabro, O. A'Amar, S. K. Singh, and I. J. Bigio, Analysis of changes in reflectance measurements on biological tissues subjected to different probe pressures. J Biomed Opt, 2008. 13(1): p. 010502.

[6] Ti, Y. and W. C. Lin, Effects of probe contact pressure on in vivo optical spectroscopy. Opt Express, 2008. 16(6): p. 4250-62.

[7] Nichols, M. G., E. L. Hull, and T. H. Foster, Design and testing of a white-light, steady-state diffuse reflectance spectrometer for determination of optical properties of highly scattering systems. Appl Opt, 1997. 36(1): p. 93-104.

[8] Marin, N. M., N. MacKinnon, C. MacAulay, S. K. Chang, E. N. Atkinson, D. Cox, D. Serachitopol, B. Pikkula, M. Follen, and R. Richards-Kortum, Calibration standards for multicenter clinical trials of fluorescence spectroscopy for in vivo diagnosis. J Biomed Opt, 2006. 11(1): p. 014010.

[9] Palmer, G. M. and N. Ramanujam, Monte Carlo-based inverse model for calculating tissue optical properties. Part I: Theory and validation on synthetic phantoms. Appl Opt, 2006. 45(5): p. 1062-71.

[10] Yu, B., D. W. Kim, J. Deng, H. Xiao, and A. Wang, Fiber Fabry-Perot sensors for detection of partial discharges in power transformers. Appl Opt, 2003. 42(16): p. 3241-50.

[11] Yu, B., A. Wang, G. Pickrell, and J. Xu, Tunable-optical-filter-based white-light interferometry for sensing. Opt Lett, 2005. 30(12): p. 1452-4.

[12] Xu, J., G. R. Pickrell, B. Yu, M. Han, Y. Zhu, X. Wang, K. L. Cooper, and A. Wang. Epoxy-free high temperature fiber optic pressure sensors for gas turbine engine applications in Sensors for Harsh Environments. 2004: Proc. of SPIE. Vol. 5590.

[13] Prahl, S., Mie scattering program. 2005, Oregon Medical Laser Center, available at http://omlc.ogi.edu/software/mie/index/html.

Thus, it can be seen that the objects of the invention have been satisfied by the structure and its method for use presented above. While in accordance with the Patent Statutes, only the best mode and preferred embodiment has been presented and described in detail, it is to be understood that the invention is not limited thereto or thereby. Accordingly, for an appreciation of the true scope and breadth of the invention, reference should be made to the following claims.

What is claimed is:

1. A smart fiber optic sensor comprising:
a sensing channel for illuminating a specimen and for collecting spectral reflections from the specimen from which specimen spectral data can be determined;
a pressure sensing channel for collecting pressure sensor spectral reflections from which a contact pressure can be determined;
a temperature sensing channel for collecting temperature sensor spectral reflections from which a temperature of the specimen can be determined; and
a calibration channel for obtaining calibration spectral reflections usable for correcting the specimen spectral data.

2. The sensor according to claim 1, wherein the temperature sensing channel comprises a fiber optic temperature sensor.

3. The sensor according to claim 2, wherein the fiber optic temperature sensor comprises an interferometric temperature sensor.

4. The sensor according to claim 3, further comprising a processing unit for calculating the temperature of the specimen based on a change in cavity length of the interferometric temperature sensor from the temperature of the specimen.

5. The sensor according to claim 1, further comprising a robotic surgical system configured to moveably carry a probe relative to the specimen, the probe being in operative communication with the sensing channel, the pressure sensing channel, the temperature sensing channel, and the calibration channel.

6. A method for utilizing a smart fiber optic sensor for measuring a specimen, the method comprising:
contacting the specimen with the smart fiber optic sensor;
generating, using the smart fiber optic sensor, specimen spectral data, pressure sensor spectral data, temperature sensor spectral data and calibration spectral data;
calculating a contact pressure at an interface of the smart fiber optic sensor and the specimen using the pressure sensor spectral data;
calculating a temperature at the interface using the temperature sensor spectral data; and
correcting specimen spectral data using the calibration spectral data.

7. The method according to claim 6, further comprising analyzing and storing the specimen spectral data.

8. The method according to claim 6, wherein generating the specimen spectral data comprises:
transmitting a first illumination light via at least one illumination fiber from a first light source to the specimen;
collecting spectral reflections at a spectrometer using at least one detection fiber, the specimen spectral reflections comprising the first illumination light diffusely reflected from the specimen at one or more wavelengths; and
generating, using the spectrometer, the specimen spectral data based on the reflections.

9. The method according to claim 6, wherein generating the calibration data comprises:
transmitting calibration light to a reflector via at least one calibration source fiber, wherein the calibration light and the first illumination light are generated simultaneously from the first light source;
collecting calibration spectral reflections at a spectrometer associated with the calibration light using at least one calibration return fiber; and
generating, using the spectrometer, the calibration spectral data from the calibration reflections.

10. The method according to claim 6, wherein generating the temperature sensor spectral data comprises:
transmitting a second illumination light using at least one optical fiber to a Fabry-Perot interferometric temperature sensor; and
collecting temperature sensor spectral reflections reflected by the temperature sensor at the spectrometer via the same optical fiber; and
generating, using the spectrometer, the temperature sensor spectral data based on the temperature sensor spectral reflections.

11. The method according to 10, wherein calculating the contact temperature comprises:
determining the cavity length of the temperature sensor using the temperature sensor spectral data;
calculating a temperature of the temperature sensor/specimen interface using the cavity length of the temperature sensor.
